(19) United States
(12) Patent Application Publication
(10) Pub. No.: US 2008/0113400 A1
(43) Pub. Date: May 15, 2008

(54) Methods and Devices for Culturing Stem Cells

(76) Inventors: Charli Kruse, Herrnburg (DE); Gunter Fuhr, Berlin (DE)

Correspondence Address: Pearl Cohen Zedek Latzer, LLP, 1500 Broadway, 12th Floor, New York, NY, 10036, US

(21) Appl. No.: 11/597,309

(22) PCT Filed: May 9, 2005
(86) PCT No.: PCT/EP05/04998

§ 371 Date: Sep. 10, 2007

(30) Foreign Application Priority Data
May 21, 2004 (DE) 10 2004 025 085.3

(51) Int. Cl. C12N 5/06; C12Q 1/02; C12M 1/00

(52) U.S. Cl. 435/29; 435/277; 435/325; 435/348; 435/349; 435/303.1

(57) ABSTRACT

Methods are described for the cultivation of stem cells (2, 3, 5), comprising the steps: Introduction of cell material comprising at least one stem cell (2, 3, 5) of a donor organism into a host egg (4) with a nutrient reservoir, and cultivation of the cell material in the host egg (4), a cell culture, and a cell culture device for carrying out the method.

### SUBJECT MATTER OF THE INVENTION

The present invention relates to methods for cultivating biological cells, especially stem cells, cell cultures containing stem cells and cell culture devices for cultivating biological cells, especially stem cells.

### BACKGROUND OF THE INVENTION

Numerous methods for the cultivation of biological cells are generally known in biology and medicine. The cultivation comprises a predetermined multiplication of cells under certain cultivation conditions. The multiplication comprises the growth of cell material, where a differentiation of cells optionally takes place. The cultivation also generally includes the influencing of the cell material in order to form differentiated cells, cell aggregates, tissues, organs or organisms.

The adjusting of the cultivation conditions in surface cultures in which the cells are arranged on a substrate and are covered with a cultivation medium is known. Surface cultures do have advantages as concerns the possibility for observing and removing cells. However, it is disadvantageous that a cell growth on solid surfaces does not occur in nature as a rule so that the cultivation conditions of the surface culture do not correspond to the natural growth conditions of cells.

The cultivation of cells in a non-adherent state, e.g., in agitated cultures or in hanging drops is also known from cultivation technology. However, these techniques have limitations as regards the adjustment of the cultivation conditions and the achievable dimensions of the cultivated cell material.

In order to simulate natural growth conditions, DE 101 09 641 A1 describes an incubator for receiving nest tissue to be used as synthetic uterus. The use of this incubator is problematic from an ethical viewpoint as well as also unsuitable for other cultivation tasks on account of the complex technical construction.

The use of bird eggs for multiplying viruses is known. However, this application is limited to a numeric multiplication of the viruses.

### OBJECT OF THE INVENTION

The invention has the object of providing improved methods for cultivating biological cells, in particular stem cells, with which the disadvantages of the traditional techniques are overcome and that in particular enable a cultivation of cells under cultivation conditions that are adapted as well as possible to natural growth conditions. The invention also has the object of providing a new type of culture in which the cells to be cultivated propagate and optionally differentiate under conditions that are as close to nature as possible. The invention also has the object of providing an improved cell culture device with which the disadvantages of traditional cultivation techniques are overcome.

These objects are achieved with cultivation methods, cell cultures and cell culture devices with the features of claims 1, 31 and 44. Advantageous embodiments and applications of the invention are derived from the dependent claims.

### DESCRIPTION OF THE INVENTION

As concerns the method, the invention is based on the recognition of the inventors that the cultivation conditions for stem cells resemble the natural growth conditions most closely, especially with respect to the geometric conditions and the nutrient supply, if the cultivation takes place in an egg. The egg generally comprises an egg cell and at least one oolemma. A natural nutrient reservoir is formed by these components. According to the invention an egg from a host organism (host egg in the following) is used for cultivation that differs from the donor organism from which the cell material introduced into the host egg stems. No exclusive nucleus transfer into the host egg takes place. The host egg for foreign stem cells or materials formed from them can surprisingly be used as cultivation substrate in which a growth and a differentiation of the at least one stem cell take place.

An essential advantage of the invention is the fact that a stem cell cultivation in the host egg enables a predetermined differentiation and an ordered growth of tissues, organs or organisms. In particular, embryos for fish farming or animal husbandry can be produced and raised or organ tissues from higher life forms can be differentiated.

Since the host egg must contain its own nutrient reservoir, according to a preferred embodiment of the invention eggs from host organisms are used as cultivation substrate that do not belong to the class of mammals. In mammals the nutrient supply during embryonic development takes place directly from the mother, so that mammalian eggs do not contain any nutrient reservoir of their own sufficient for a rather long embryonic development. For the rest, the host organism can be selected depending on the concrete cultivation object and the donor organism. According to the invention host eggs can advantageously be selected from the taxonomic classes of birds, fish, amphibians, reptiles and insects, whose eggs all have a sufficient nutrient reservoir. The donor and host organisms belong with particular preference to different taxonomic units, in particular classes, families or species in order to keep the influence of foreign cells on the stem cells to be cultivated as low as possible. Preferred applications of the invention consist in the introduction of cell material stemming from mammalian organisms into bird eggs or reptile eggs or in the introduction of cell material stemming from fish of a first species into fish eggs of a different species.

If according to another embodiment of the invention an unfertilized host egg is used for cultivating the cell material, exclusively the foreign cells can advantageously propagate therein. Alternatively, the foreign cell material can be introduced into an already fertilized host egg, which can yield advantages for the development of an optimal nutrient reservoir in the host egg. In this embodiment of the invention, the germ disk possibly developing in the host egg is removed or deactivated in order not to disturb the further growth of the foreign cell material.

According to another preferred embodiment of the invention the cell material introduced into and cultivated in the host egg comprises one or more adult stem cells, in particular adult multipotent or pluripotent stem cells stemming from an exocrine gland of the donor organism.

It was observed that pluripotent stem cells with a high capacity for growth and differentiation can surprisingly be obtained from exocrine glands of adult organisms that tolerate the cultivation in the host egg of a foreign organism particularly well on account of their strong and stable capacity for growth. The at least one stem cell cultivated in the host egg preferably stems from exocrine glandular tissue from a vertebrate, e.g., a fish, amphibian, reptile, bird or mammal, especially preferably from a primate, in particular a human.

The exocrine glandular tissue used for obtaining stem cells for the cultivation in accordance with the invention can stem from an adult organism, juvenile organism or non-human fetal organism, preferably a postnatal organism. The concept “adult” as it is used in the present application thus refers to the development stage of the initial tissue and not to that of the donor organism from which the tissue stems. “Adult” stem cells are non-embryonic stem cells.

The cell material cultivated in accordance with the invention is preferably isolated from exocrine glandular tissue from a salivary gland, lachrymal gland, sebaceous gland, sweat gland, from glands of the genital tract including the prostate, or from gastrointestinal tissue, including the pancreas, or from secretory tissue of the liver. In a very preferred embodiment acinar tissue is concerned. The acinar tissue stems especially preferably from the pancreas, the parotid gland or the submandibular gland.

Advantageously, the stem cells provided from these donor tissues can be readily isolated and maintained in a non-differentiated state in a stable long-time culture without a feeder cell layer or special additives. The concept feeder cells as it is used in the present application comprises all cells that promote the growth of the cells that are actually to be cultivated in that they release growth factors and/or provide an extracellular matrix or prevent the differentiation of the stem cell culture.

In stem cell cultures of the inventors the cells retained their ability for self-renewal and unlimited division for a year after more than 25 previous passages and the cultures continue to be stable. This property of adult pluripotent stem cells from exocrine glandular tissue is especially advantageous for the adjustment of defined start conditions in the cultivation method in accordance with the invention.

The adult stem cells used in accordance with the invention can be induced to differentiate in a simple manner without the addition of special growth or differentiation factors in that they are cultivated in the host egg under spatial conditions that ensure a three-dimensional contact of the cells.

According to a modified embodiment of the invention, cell material that contains at least one non-human embryonic stem cell is cultivated in the host egg. Non-human embryonic stem cells are advantageously pluripotent and thus suitable when cultivating for differentiations into all germ layers.

According to a further modification, the cell material injected in accordance with the invention into the host egg may comprise a plurality of stem cells in that form a cohesive aggregate (or: cell bodies or tissue bodies). In this instance, advantageously the chances can be increased that the vitality of the material remains preserved during the cultivation. Furthermore, the cell bodies or tissue bodies may be pre-differentiated so that a certain direction of differentiation can be impressed on the cultivation in accordance with the invention in the host egg.

The formation of the organoid bodies takes place in a pre-cultivation under cultivation conditions in which a three-dimensional contact of the stem cells is also given. In a preferred embodiment the pre-cultivation takes place in hanging drops such as has already been described for embryonic stem cells (Wobus et al., Biomed. Biochim. Acta 47:965-973 (1988)). This method will be described in more detail in the following in the examples. It is understood, however, that alternative cultivation methods that ensure the desired three-dimensional contact of the cells and are known and available to an expert in the art can also be used. Examples of such alternative methods are the cultivation in a moved suspension culture, the cultivation in an electromagnetic field cage or laser tweezer, the seeding of non-resuspended primary cells on culture plates or the cultivation on surfaces to which the cells do not adhere or adhere only poorly. Such surfaces can be, e.g., glass, polystyrene or surfaces treated with an anti-adhesion layer, e.g., surfaces coated with PTFE or poly-HEMA.

During the pre-cultivation three-dimensional cell compounds or cell aggregates spontaneously develop from adult pluripotent stem cells, which compounds or aggregates are also referred to as “organoid bodies”. These organoid bodies can be transferred into suspension cultures or adhesion cultures and further cultivated. Given a sufficient supply of nutrients, the organoid bodies continue to grow and can achieve diameters of a few millimeters or more. The large organoid bodies exhibit a tissue-like structure and are also referred to in this stage as “tissue bodies” in order to distinguish them from the simple cell aggregates.

If the organoid bodies are brought back into surface culture, a cellular monolayer is produced from out-growing individual cells from which monolayer multi-layer areas arise from which secondary organoid bodies are spontaneously formed with properties comparable to those of the primary organoid bodies.

The organoid bodies that can be used in accordance with the invention can be stored frozen, e.g., at -the temperature of liquid nitrogen, without losing their viability and their ability to reproduce, grow and differentiate.

The formation of embryoid bodies from non-human embryonic stem cells takes place during the pre-cultivation under analogous cultivation conditions, especially like those already described for embryonic stem cells (see above, Wobus et al.).

The introduction or injection of the cell material into the host egg can take place with known methods suitable for this purpose such as, e.g., an injection of suspended cell material with a capillary or syringe.

According to a preferred embodiment of the invention a differentiated growth of the cell material takes place in the host egg. Different stem cells differentiate in different directions depending on the geometric conditions and possibly on externally influenced material conditions in the host egg. Cells that grow from the originally injected stem cells, organoid bodies or tissue bodies can differentiate into the different cell types of all three germ layers. No differentiation factors are necessary for the differentiation and the cells also do not have to be transplanted in order to differentiate. However, it can be advantageous to use such differentiation factors in order to purposefully produce larger amounts of a certain cell type. Such differentiation factors are known in the state of the art and comprise, e.g., bFGF (basic fibroblast growth factor) for an increased formation of cardiac cells and fibroblasts, VEGF (vascular endothelial growth factor), DMSO and isoproterenol, fibroblast growth factor 4 (FGF4), hepatocyte growth factor (HGF) for an increased formation of cardiac and liver cells, TGF-betal (transforming growth factor betal) for an increased formation of cardiac cells, EGF (epidermal growth factor) for an increased formation of skin cells and cardiac cells, KGF (keratinocyte growth factor) (sometimes together with cortisone) for the formation of keratinocytes, retinoic acid for an increased formation of nerve, cardiac and kidney cells, beta NGF (beta nerve growth factor) for an increased formation of brain, liver, pancreatic and kidney cells, BMP 4 (bone morphogenic protein 4) and activin A for the formation of mesodermal cells in general, but are not limited to them.

Differentiated cells obtainable from the stem cells used in accordance with the invention comprise bone cells (osteoblasts and osteoclasts), chondrocytes, adipocytes, fibroblasts (e.g., skin- and tendon fibroblasts), muscle cells, endothelial cells, epithelial cells, hematopoietic cells, sensory cells, endocrine and exocrine glandular cells, glia cells, neuronal cells, oligodendrocytes, blood cells, intestinal cells, cardiac, lung, liver, kidney or pancreatic cells, but are not limited to them.

According to an especially preferred embodiment of the invention the differentiated growth can be influenced externally by the adjusting of material, mechanical and/or thermal growth conditions. For example, a temperature-adjusting of the host egg can be provided in order to achieve a cultivation corresponding to the breeding of a bird egg. Mechanical growth conditions comprise, e.g., a moving of the host egg or a predetermined geometric alignment in a gravitational field. The adjustment of material growth conditions preferably comprises the injection of at least one differentiation and/or growth factor into the host egg.

If at least one property of the cell material is measured during its cultivation, this can result in special advantages for the monitoring and influencing of the course of growth. The measuring preferably detects if the cultivated cell material is still alive. The measuring of vitality comprises, e.g., a measuring with NMR or ultrasound. Alternatively or additionally a detection of a degree of differentiation of the cell material can be provided. The cultivation conditions can be advantageously changed depending on the result of the measurement.

The invention provides in accordance with a further variant that the cultivation of the cell material in the host egg is terminated when a predetermined cultivation state has been achieved. This termination can prevent any cell material that is misdifferentiated or proliferating in an undesired manner from further growth. The cultivation conditions employed for terminating the cultivation preferably comprise the cultivation time, the attained size of the cultivated material and/or the attained degree of differentiation of the cultivated cell material.

A further modification can provide that cultivated cell material is removed from the egg without the oolemma being damaged. For example, differentiated cell material can be continuously removed from the egg.

According to the invention the stem-cell-based cell material can be arranged at one or several positions in the host egg. The injection of the cell material is preferably at the location in the host egg that corresponds to the natural position of the germ disk in the uninfluenced egg. This embodiment of the invention is especially advantageous since the cultivation begins under geometric and material conditions that resemble the natural incubation conditions most closely.

The cellular material is preferably positioned in a cultivation bubble in the host egg which bubble contains at least one liquid or gelatinous cultivation substance. This permits a purposeful setting of material cultivation conditions in the host egg. According to preferred variants of the invention the cultivation substance in the cultivation bubble can be replaced continuously or according to pre-settable cultivation protocols at certain time intervals, e.g., periodically. The cultivation substance can also be arranged in the vicinity of the cell material at the location of the nutrient reservoir of the host egg in that the nutrient reservoir (in particular the egg yolk, egg white) is displaced or at least partially replaced. The advantage of this embodiment of the invention is that the geometric cultivation conditions in the host egg remain unchanged but the material cultivation conditions can be adapted to the requirements of the donor organism. The cultivation substance can be renewed or replaced even in the nutrient reservoir during the cultivation of the cell material on a continuous basis or in pre-settable time intervals.

It can be provided in accordance with the invention that the egg white of the host egg is completely replaced by the cultivation substance. Furthermore, a complete replacement of the egg yolk can be provided, where, however, the geometric arrangement of both components and the yolk covering remain preserved.

The replacement preferably comprises an introduction of drops of the cultivation substance into the nutrient reservoir of the host egg. The liquid or gelatinous drops can comprise a membrane covering through which active substances, e.g., hormones or differentiation factors can diffuse into the egg. For example, a lipid membrane is provided.

If, according to another embodiment of the invention, certain material and/or temperature gradients are generated during the cultivation of the cell material in the host egg, the cultivation of the cell material can advantageously take place according to pre-settable material and/or geometric cultivation protocols. A particular advantage of natural host eggs is that a material as well as a thermal influence can be exerted on the interior of the cell material through the oolemma even in the formation of a lime shell.

According to the invention it is also possible to cultivate the cell material in the host egg in the presence of at least one solid. Geometric limitations for the stem cell cultivation and differentiation can advantageously be set with the solid. If, e.g., at least one carrier particle is used as solid, it can form a substrate that is grown around during the cultivation. This application of the invention is advantageous for the production of implant materials. Furthermore, solids can also be arranged at first in a distance from the cellular material in order to form a geometric limitation during its growth. Such geometric limits can be generated even in the mm to cm range. To this end, e.g., liquids are introduced into the host egg in a liquid state (e.g., liquid polymers) and then hardened locally, e.g., under the influence of heat or light.

According to an independent aspect of the invention, the above-cited object is achieved regarding products by a cell culture comprising a composition of a host egg and at least one stem cell arranged in the host egg. The donor organism from which the adult pluripotent or non-human embryonic stem cell was obtained is not identical with the host organism from which the host egg was obtained. The host organism and the donor organism preferably even belong to different taxonomic classes.

As concerns the device, the above-mentioned object is achieved by providing a cell culture device including an incubator device set up to receive an egg from a host organism. To this end, the incubator device has an incubator chamber that is greater than the host egg. The inner form of the incubator chamber is preferably adapted at least in partial areas to the outer form of the host egg. The cell culture device in accordance with the invention advantageously has an especially simple design since merely a receptacle for the host egg with the cell material to be cultivated has to be provided. In distinction to traditional incubator devices the cell culture device in accordance with the invention requires no separate cultivation substrate since the latter is formed by the host egg that can be placed into the incubator chamber.

Advantages for a broad and flexible usage of the cell culture device in accordance with the invention result if it is equipped with an injection device for introducing solutions or suspensions into the interior of the host egg, with a temperature-adjusting device for adjusting the temperature of the incubator chamber, a humidifier for adjusting a predetermined humidity in the incubator chamber, a measuring device for detecting properties of the host egg or of the cultivated cell material, and/or a drive device for moving the host egg in the incubator device. The injection device comprises, e.g., a cell injector for introducing suspensions of cells or cell aggregates such as, e.g., cell bodies. The cell injector preferably has a rigid injection line, e.g., an injection needle with which a deposition of cell material can take place in the host egg at predetermined positions. In order to introduce growth or differentiation factors, the injection device furthermore includes a liquid injector. The cell injector and the liquid injector are preferably formed by a common component.

Independent subject matter of the invention is constituted by the use of an egg of a host organism for cultivating biological cells that do not stem from the host organism. The use of an egg, especially a bird or fish egg, as incubator for stem cells introduced from the outside into the egg is especially preferred.

A preferred application of the invention consists in the development of multicellular systems that are similar to tissues or organs in vitro. In a preferred embodiment the multi-cellular systems comprise different cell types. These multi-cellular systems can then be, e.g., transplanted or used extracorporally, e.g., for blood washing or for the production of desired substances or as pharmacological tissue model.

### BRIEF DESCRIPTION OF THE FIGURES

Other details and advantages of the invention are described in the following with reference made to the attached drawings.

FIG. 1 shows a flowchart for illustrating steps of the method in accordance with the invention.

FIG. 2 schematically shows the transfer of stem cells into a bird egg.

FIG. 3 shows individual steps in the preparation of cell material.

FIG. 4 schematically shows different variants of the transfer of cell material into a bird egg.

FIG. 5 shows individual steps of the deposition of cell material in the host egg.

FIG. 6 shows further details of the depositing of cell material and additives in a bird egg.

FIG. 7 shows individual steps of the cultivation of cell material in the host egg.

FIG. 8 schematically shows a sectional view of a cultivation device in accordance with the invention.

FIG. 9 schematically shows the cultivation of stem cells in surface culture and in hanging drops as well as the formation and further cultivation of organoid bodies.

### DESCRIPTION OF PREFERRED EXEMPLARY EMBODIMENTS

Reference is made by way of example in the following description of preferred exemplary embodiments of the invention to the cultivation of adult pluripotent stem cells in a host egg. The invention can be used analogously in the cultivation of non-human embryonic stem cells, their obtention and optional pre-cultivation to embryoid bodies being known and not described here.

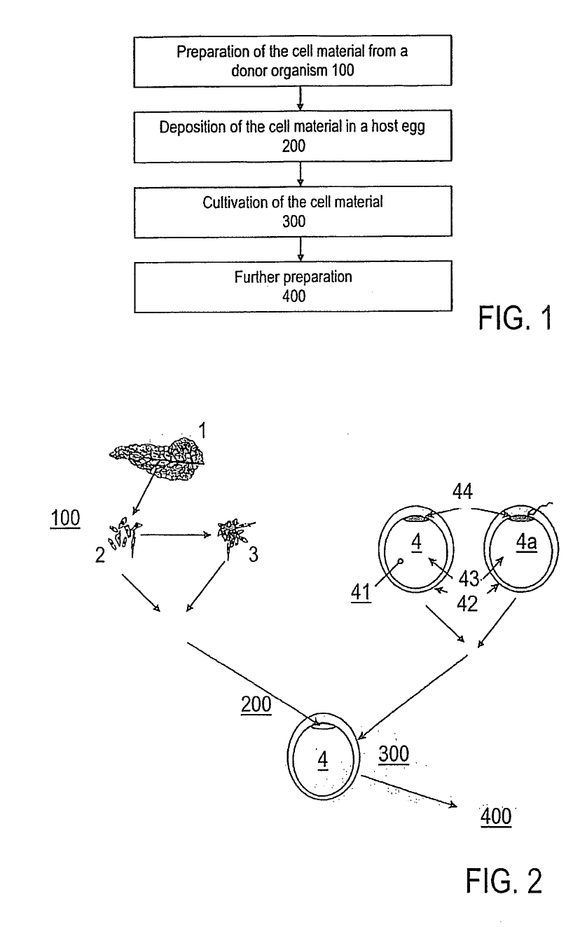

In general, the method in accordance with the invention comprises the introduction and cultivation of at least one stem cell into a host egg of a foreign host organism. The realization of this method including optionally provided preparatory and follow-up steps is schematically illustrated in FIG. 1 and further details are explained below.

At first, the preparation of the cell material that comprises at least one stem cell and is to be introduced into the host egg takes place in step 100. The preparation comprises the isolation of the stem cells from the donor organism and optionally their pre-cultivation to organoid bodies or tissue bodies described below with further details. Subsequently, the cell material is deposited in the host egg in step 200. The growth and the differentiation of the stem cells (cultivation in step 300) begins immediately after the introduction and deposition. Further preparation procedures or processing procedures may follow depending on the cultivation protocol in step 400.

Further details of the steps 100 to 400 are illustrated in FIG. 2. The preparation step 100 comprises the isolation of the stem cells 2 from the exocrine glandular tissue, e.g., the pancreas 1 of a mammal, e.g., a mouse. The stem cells 2 from the primary culture can then be introduced in step 200 into the host egg. It is alternatively possible to first form the organoid bodies or tissue bodies 3 from the stem cells 2 by a pre-cultivation and aggregation, which bodies are then introduced into the host egg.

The host egg 4 is, e.g., a bird, reptile, or fish egg having an egg cell 41 and at least one oolemma 42. The egg cell 41 comprises egg yolk 43 and the germ disc 44. The right part of FIG. 2 illustrates that unfertilized eggs 4 or already fertilized eggs 4a can be alternatively used for the cultivation of stem-cell-based cell material in accordance with the invention.

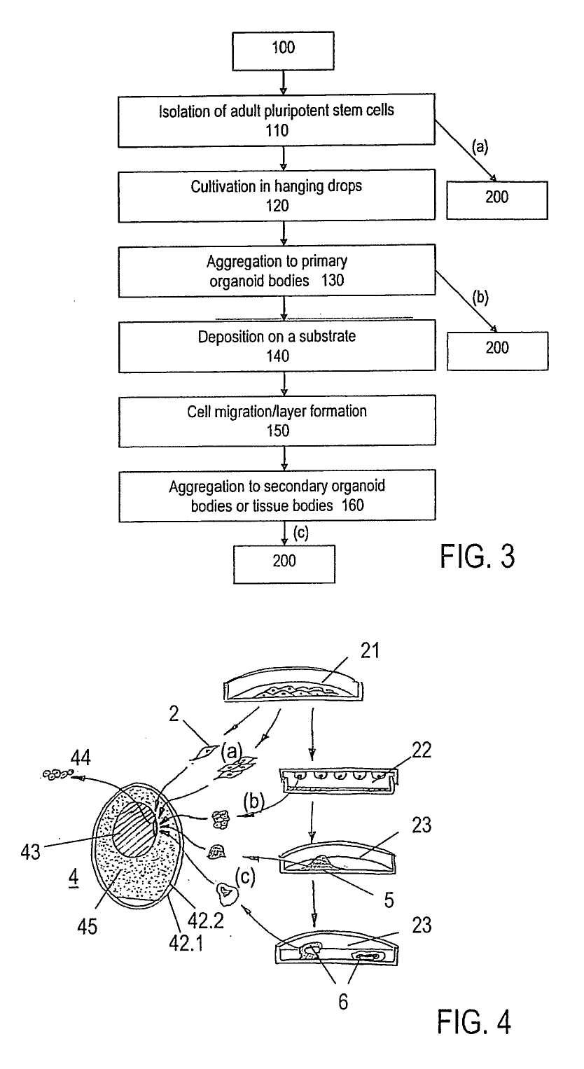

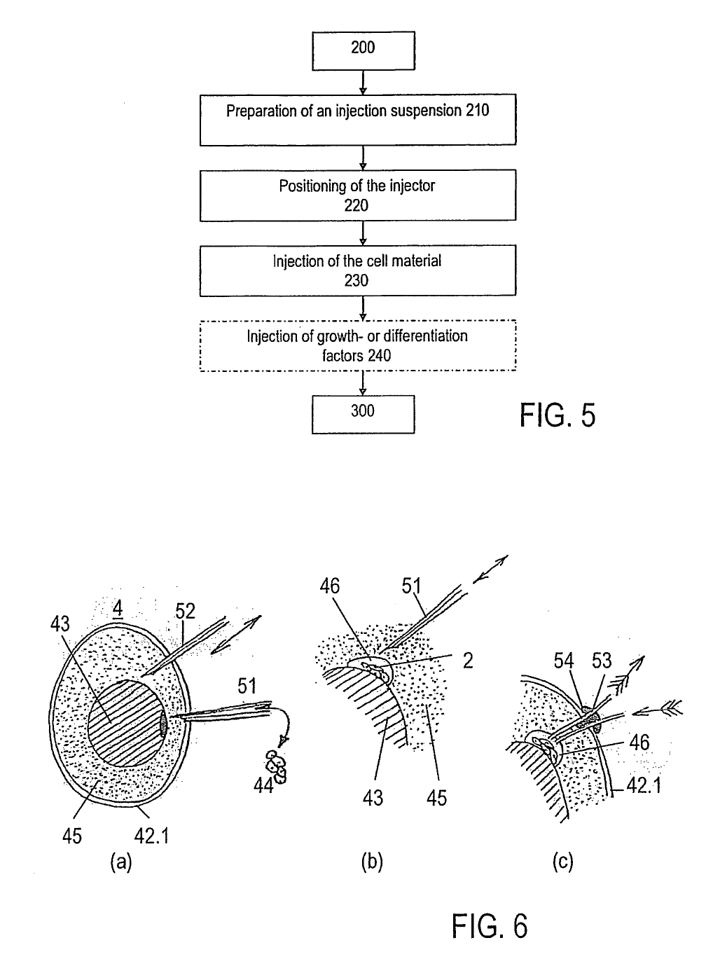

FIG. 3 shows the different options for providing stem cells, organoid bodies or tissue bodies as starting materials for the cultivation. At first, the isolation of adult pluripotent stem cells from a donor organism is provided in step 110. According to the first variant (a) the deposition 200 of the cell material in which the primarily obtained stem cells are introduced into the host egg takes place. Alternatively, the cultivation of the isolated stem cells in hanging drops described below with further details (step 120; see FIG. 5) with the aggregation to the so-called primary organoid bodies (step 130) takes place next. According to variant (b) the primary organoid bodies can be deposited directly for cultivation in the host egg (step 200 in FIG. 1). According to a further alternative the deposition of the primary organoid bodies on a substrate (step 140) for creating an adhesion culture (see also FIG. 4) takes place next. The inventors observed that the primary organoid bodies growing in suspension culture in an adhesion culture grow to larger tissue bodies or can form new organoid bodies. The formation of a monolayer on the substrate by a cell migration (step 150) follows and from this monolayer the aggregation to the so-called secondary organoid bodies (step 160). According to variant (c) the latter can be directly deposited again for cultivation (step 200) and used.

Variants (a) to (c) are also illustrated in FIG. 4. The host egg 4 is, e.g., a chicken egg with a nutrient reservoir of egg yolk 43 and egg white 45. The nutrient reservoir is surrounded by a lime shell 42.1 that together with an egg membrane 41.2 forms the oolemma. The germ disk 44 is located on the boundary surface between the egg yolk 43 and the egg white 45. The germ disk 44 is removed from the host egg 4 in a preparatory step. This takes place with mechanical means, e.g., by suction removal with a cannula. Alternatively, the germ disk 44 can be deactivated by the addition of a chemical substance (e.g., with a lethal dye known from microscopy or hydroxyl urea) or by radioactive irradiation (e.g., with y-rays). The stem-cell-based cell material is subsequently deposited in the host egg 4 at the location of the natural position of the germ disk. According to variant (a) individual stem cells 2 or a plurality of stem cells 2 are introduced directly from primary culture 21 into the host egg 4. The positioning preferably takes place below or above the vitellin membrane of the egg yolk without destroying this membrane. If the hanging drop culture 22 is formed at first (step 120 in FIG. 3), cell aggregates from the hanging drop culture 22 according to variant (b) can be deposited in the host egg 4. The deposition of the secondary organoid bodies 5 formed in the adhesion culture 23 is illustrated in variant (c). When the primary organoid bodies grow to tissue bodies 6 the latter can also be removed from the adhesion culture 23 and introduced into the host egg 4. The tissue bodies 6 have been produced by a growth of organoid bodies in the adhesion culture and have a characteristic diameter of a few hundred micrometers to a few millimeters.

The preparation and pre-cultivation of non-human embryonic stem cells and embryoid bodies take place analogously to the illustrated variants (a) to (c).

Further details of the deposition step 200 according to FIG. 1 are compiled by way of example in FIG. 5. After preparation of the cell material to be cultivated an injection suspension is formed at first in step 210. The injection suspension comprises the cell material, e.g., a plurality of stem cells or organoid bodies and a carrier liquid. The carrier liquid is, e.g., a physiological nutrient solution (e.g.: HEPES's Eagle medium, DMEM incubation medium or the like). In step 210 the association of the cell material with substrates or carrier particles, e.g., plastic beads or glass beads that are to be introduced as solid matter with the cell material into the host egg can be provided. The injector (see FIG. 6) is subsequently positioned for the introduction of the cell material in step 220. The injector is moved through the oolemma and the nutrient reservoir up to the desired deposition position at the location of the germ disk. For example, an injection needle is aligned with a positioning device relative to the desired deposition position and introduced into the host egg with a drive device. The correct positioning of the injector can be checked, e.g., with an ultrasonic measurement. The injection of the cell material subsequently takes place with the injector in step 230. Further growth and/or differentiation factors can then be introduced in step 240 into the host egg in the immediate vicinity of the injected cell material depending on the cultivation object.

FIG. 6 illustrates various phases of the preparation and charging of the host egg 4. The partial image (a) shows the egg 4 with the egg yolk 43, the egg white 45 and the germ disk 44 inside the lime shell 42.1. A suspension injector 51 and a liquid injector 52 are shown by way of example that are each formed by a capillary or injection needle and can be moved in a direction along their longitudinal extent (see double arrow). The germ disk 44 can be removed at first with the suspension injector 51. The suspension injector 51 is subsequently used for the above-described introduction of the cell material. The liquid injector 52 serves, e.g., for the transfer of other components affecting the cultivation of the cell material such as, e.g., growth and/or differentiation factors. These components can be supplied during the preparation of the host egg 4 before the cultivation and/or during the cultivation of the cell material in the host egg 4.

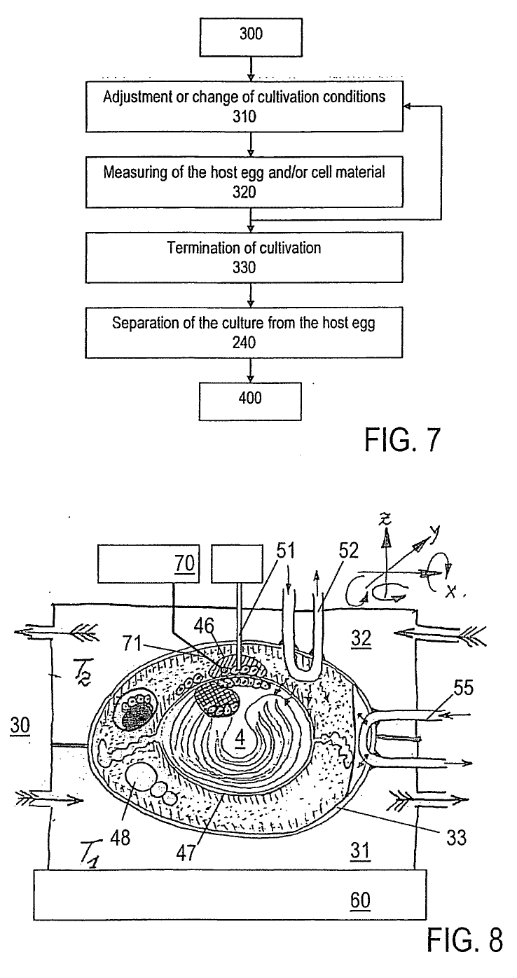

The removal and introduction of cells or chemical substances take place in that a hole is formed in the line shell 42.1 with the tip of the injector. The formation of the hole and further actuation of the injectors takes place under sterile conditions. Partial image (b) of FIG. 7 shows the section in the interior of the host egg 4 in which the stem cells 2 were arranged with the suspension injector 51 at the boundary between the egg yolk 43 and the egg white 45 at the location of the previously removed germ disk. The stem cells 2 are in the cultivation bubble 46 formed by the suspension liquid and optionally containing growth and/or differentiation factors.

Partial image (c) shows the supplying of the cultivation bubble 46. According to the invention one or more liquid lines 53 can run from the cultivation bubble 46 to the outside during the cultivation (see FIG. 8) in order to supply cultivation medium or to remove metabolic products. The liquid lines 53 are run through an opening in the lime shell 42.1 closed by a seal 54. The seal 54 consists, e.g., of paraffin or some other plastically deformable polymer.

A flow of solution through the cultivation bubble 46 can be provided via the liquid lines 53 with which, e.g., the volume of the cultivation bubble can be reduced or increased according to pre-settable cultivation procedures. Furthermore, altered cultivation solutions, e.g., with changing additives (e.g., hormones, differentiation factors) can be flushed in or further biological cells such as, e.g., stem cells or pre-differentiated cells can be introduced via the liquid lines 53. The removal of cultivated cell material from the cultivation bubble 46 is also possible in a corresponding manner.

FIG. 7 illustrates further details of step 300 of the cultivation of the cell material (see FIG. 1). The adjustment of cultivation conditions in the host egg 4 takes place after the deposition of the cell material in step 200 if this did not take place already in the framework of a preparation of the host egg. The cultivation conditions are selected to be as physiological or as close to nature as possible. Thus, e.g., a cultivation temperature corresponding to the typical body temperature of the donor organism is selected. Furthermore, a movement of the host egg can be provided that corresponds to the rotating movement on bred eggs practiced by birds.

At least one measurement on the host egg 4 and/or on the cultivated cell material is provided in step 320 in order to determine the progress of the cultivation or the attainment of a certain cultivation state. The measured result is compared with a given cultivation protocol. The cultivation is optionally terminated depending on the comparison (step 330). Alternatively, the cultivation is continued under the given conditions or under changed cultivation conditions (return to step 310). Finally, a separation of the cultivated cells from the host egg takes place in step 240 after the termination of the cultivation.

FIG. 8 shows an exemplary embodiment of a cell culture device in accordance with the invention. The cell culture device comprises an incubator device 30 with a bottom part 31 and a cover part 32 that form an incubator chamber 33 in the assembled state for receiving the host egg 4. The bottom and cover parts 31, 32 have several functions. In addition to the formation of the incubator chamber 33 they also serve for temperature-adjustment and optionally for the exchange of materials. The bottom and cover parts 31, 32 form a temperature-adjusting device and/or a humidifier. To this end, the bottom and cover parts 31, 32 each are hollow components that can be filled with temperature-adjusting liquids and/or supply liquids (or corresponding gases) or which liquids can flow through them. In order to achieve the exchange of materials, the bottom and cover parts 31, 32 comprise a semi-permeable wall at least in partial areas on the sides facing the incubator chamber 33 through which wall the substances can diffuse into the host egg.

Furthermore, the cell culture device is provided with a drive device 60 with which the incubator device 20 can be pivoted in all spatial directions (see coordinate system). The drive device 60 comprises, e.g., a pivot drive known from laboratory technology and/or rotary table drives.

The cell culture device contains other components that form the injection device 50. Parts of the injection device 50 that may be provided individually or together comprise the suspension injector 51, a liquid injector 52 and a gas injector 55. The liquid injectors and gas injectors 52, 55 comprise liquid lines and gas lines that are run through a partial area of the host egg 4 and have a semi-permeable wall at least in this partial area. As a result, substances can be introduced by diffusion with the liquid injector 52, e.g., into the egg white or with the gas injector 55 into the gas bubble of the host egg 4. The reference numeral 56 refers in a general manner to a positioning and drive device for the suspension injector 51. The positioning and drive device contains, e.g., a piezoelectric drive.

The reference numeral 70 refers in a general manner to a measuring device that is contactless or operates in contact with the host egg. The measuring device 70 for contactless operation is, e.g., an NMR measuring device or an ultrasonic measuring device. For contact operation the measuring device 70 is provided with a probe 71 with which local measurements can be carried out in the host egg 4, e.g., on the cultivated cell material. The probe 71 can be designed, e.g., for temperature measurements, electrical measurements or optical measurements (e.g., fluorescence measurements).

FIG. 8 illustrates the cultivation of the cell material in a cultivation bubble 46 above or below the membrane 47 between the egg yolk and the egg white. The cultivation which is alternatively possible without a cultivation bubble is also shown. The reference numeral 48 refers to drops with a cultivation substance that were introduced from the outside into the egg white.

### FURTHER EXAMPLES

The present invention will be explained in detail in the following non-limiting examples.

The general working instructions customary for methods for cultivating animal cells and in particular mammalian cells, are to be observed. A sterile environment in which the method is to be carried out is to be observed in any case, even if no further description for this is given. The following buffers and media were used:

| | |
|---|---|
| HEPES stock solution (pH 7.6) | 2.383 g HEPES per 100 ml A. bidest. |
| HEPES Eagle's Medium (pH 7.4) | 90 ml modified Eagle's Medium (MEM) |
| | 10 ml HEPES stock solution |
| Isolation medium (pH 7.4) | 32 ml HEPES Eagle's Medium |
| | 8 ml 5% BSA in A. bidest. |
| | 300 μl 0.1 M CaC1₂ |
| | 100 μl trasylol (200,000 KIU) |
| Digestion medium (pH 7.4) | 20 ml isolation medium |
| | 4 ml collagenase (collagenase NB 8 from Serva) |
| Incubation medium | Dulbecco's modified Eagle's Medium (DMEM) |
| Nutrient medium | Dulbecco's modified Eagle's Medium (DMEM) |
| | DMEM + 4500 mg/l glucose + L- |
| | glutamine − pyruvate + 20% |
| | FCS (inactivated) + 1 ml/100 ml |
| | pen/strep |
| | (10000 U/10000 μg/ml) |
| | or |
| | DMEM + 10% autoplasma + 1 ml/100 ml |
| | pen/strep, |
| | warm to 37° C. before use |
| Differentiation medium | 380 ml DMEM |
| | 95 ml 30 min at 54° C. inactivated FCS |
| | 5 ml glutamine (GIBCO BRL) |
| | 5 ml (3.5 μl β-mercaptoethanol per 5 ml PBS) |
| | 5 ml nonessential amino acids (GIBCO BRL) |
| | 5 ml penicillin/streptomycin (GIBCO BRL) |
| | (10000 U/10000 μg/ml) |

Instead of fetal calf serum (FCS) in the nutrient medium and differentiation medium optionally autoplasma or, less preferably, autoserum of the tissue donor may be used as well. This is particularly significant if the tissue donor is identical with the later recipient of the stem cells or of differentiated cells derived from them. Such an autologous treatment is preferred in order to prevent a possible rejection reaction.

Instead of the DMEM medium used, the nutrient medium can also contain another suitable base medium known for the cultivation of eukaryotic cells, especially mammalian cells, as base medium in which the differentiated cells die and the desired stem cells propagate. The isolation medium, incubation medium and differentiation medium may contain a different customary and suitable base medium as well.

The following examples 1 and 2 describe in detail two working protocols for the isolation and cultivation of adult pluripotent stem cells from acinar tissue of the pancreas. Example 3 describes a corresponding protocol for the isolation from acinar and tubular tissue of the salivary gland.

### EXAMPLE 1

1. Preparation of the Tissue and Isolation of the Cells

10 ml digestion medium were injected slowly and free of air bubbles into the Ductus pancreaticus of 2-3-year old rats with a syringe and blunt cannula in the rat. The entire pancreas is inflated as a result and can thus be better prepared for removal. The pancreas is then transferred into a beaker glass and 5 ml more digestion medium are added to it. After the fatty tissue and lymph nodes have been removed the tissue is comminuted very finely in the beaker glass with fine shears, fatty tissue floating on top is removed by suction and the suspension is subsequently gassed 1 min with Carbogen (repeat if necessary) and incubated in an agitator at 200 cycles/min for 20 min at 37° C. with aluminum foil. Then, the medium is carefully removed by suction, the tissue comminuted again with shears and the tissue pieces washed twice with 10 ml isolation medium each time and 5 ml digestion medium is again added to the tissue.

After another gassing with Carbogen for approximately 1 minute and incubation for 15 min at 37° C. at 200 cycles/min, the tissue pieces are comminuted by successively being drawn up in a 10 ml, 5 ml, 2 ml and 1 ml glass pipette and pressed through a monolayer filter tissue. The cells individualized in this manner are now washed five times in incubation medium (3720 C.), gassed with Carbogen and centrifuged each time for 5 min at 90 g. The last pellet obtained is resuspended in incubation medium, gassed and distributed onto tissue culture dishes.

2. Cultivation of the Cells

The tissue culture dishes with the isolated cells are cultivated in an incubator at 37° C. and 5% CO₂. The medium is replaced every 2-3 days at which time all recognizable differentiated cells are removed.

On the seventh day in culture the cells are passaged with a solution consisting of 2 ml PBS, 1 ml trypsin and 2 ml incubation medium, during which the cells separate from the bottom of the culture dish. The cell suspension is centrifuged 5 minutes, the supernatant removed by suction and the cells resuspended in 2 ml incubation medium, transferred to a medium sized cell culture bottle and 10 ml incubation medium are added thereto. The medium is replaced every three days.

On the fourteenth day in culture the cells are passaged again but this time with 6 ml PBS, 3 ml trypsin and 6 ml incubation medium. The cell suspension is centrifuged 5 minutes, the supernatant removed by suction and the cells are re-suspended in 6 ml incubation medium, transferred to 3 medium cell culture bottles and 10 ml incubation medium added to each one.

The cells are cultivated further and passaged and seeded until the cells achieve a semi-confluent to confluent state. The introduction and further cultivation in the host egg takes place in accordance with the invention in this state.

### EXAMPLE 2

Pancreas acini were obtained from male Sprague-Dawley rats (20-300 g) that had been narcotized (CO₂) and exsanguinated via the dorsal aorta. A cannula was introduced transduodenally into the pancreatic duct and 10 ml digestion medium containing HEPES Eagle's Medium (pH 7.4), 0.1 mM HEPES buffer (pH 7.6), 70% (vol./vol.) modified Eagle's Medium, 0.5% (vol./vol.) trasylol (Bayer AG, Leverkusen, Germany), 1% (wt./vol.) Bovine serum albumin), 2.4 mM CaCl₂ and collagenase (0.63 P/mg, Serva, Heidelberg, Germany) were injected into the pancreas from the rear.

Prior to the removal the pancreas was partially freed of adhering fatty tissue, lymph nodes and blood vessels. Then, healthy pancreatic tissue was taken in digestion medium (at 20° C., lesser metabolism), the pancreatic tissue very finely comminuted with shears, fatty tissue floating on top removed by suction and the tissue suspension gassed with Carbogen (Messer, Krefeld, Deutschland) without the nozzle entering into the medium with the cells (reduction of mechanical stress) and adjusted therewith to pH 7.4. The suspension was then incubated in a 25 ml Erlenmeyer flask (covered with aluminum foil) under constant agitation (150-200 cycles per minute) at 37° C. in 10 ml digestion medium. After 15-20 minutes the fat floating on top and the medium were removed by suction and the tissue was again comminuted and rinsed with medium without collagenase (repeat procedure at least twice, preferably until cell fraction transparent), whereupon digestion medium was added and another gassing was performed for approximately 1 minute with Carbogen. A digestion with collagenase followed again for 15 minutes at 37° C. in an agitator using the same buffer. After the digestion the acini were dissociated by successively drawing them up and ejecting them through 10 ml, 5 ml and 2 ml glass pipettes with narrow openings and filtered through a single-layer nylon mesh (Polymon PES-200/45, Angst & Pfister AG, Zurich, Switzerland) with a mesh size of approximately 250 μm. The acini were centrifuged (at 37° C. and 600-800 rpm in a Beckman GPR centrifuge, corresponds to approximately 50-100 g) and further purified by washing in incubation medium containing 24.5 mM HEPES (pH 7.5), 96 mM NaCl, 6 mM KCl, 1 mM MgCl₂, 2.5 mm NaH₂PO4, 0. mM CaCl₂, 11.5 mM glucose, 5 mM sodium pyruvate, 5 mM sodium glutamate, 5 mM sodium fumarate, 1% (vol./vol.) modified Eagle's Medium, 1% (wt./vol.) bovine serum albumin, equilibrated with Carbogen and adjusted to pH 7.4. The washing procedure (centrifugation, removal by suction, re-suspension) was repeated five times. Unless otherwise indicated, the work was performed at approximately 20° C. in the above isolation.

The acini were re-suspended in incubation medium and cultivated at 37° C. in a humid atmosphere with 5% CO₂. The acinar tissue died rapidly (within two days) and the dying differentiated cells separated from the adjacent cells without damaging them (gentle isolation) and the stem cells that were not dying sank to the bottom, to which they adhered. The differentiated acini cells were not capable of doing this. The incubation medium was replaced for the first time on the second or third day after the seeding, during which a large part of the freely floating acini and acinar cells was removed. At this time the first stem cells or their precursors, respectively, had attached themselves to the bottom and began to divide. The medium replacement was repeated thereafter on every third day and differentiated acinar pancreatic cells were removed at each medium replacement.

On the seventh day in culture the cells were passaged with a solution consisting of 2 ml PBS, 1 ml trypsin (+0.05% EDTA) and 2 ml incubation medium, during which the cells separated from the bottom of the culture dish. The cell suspension was centrifuged 5 minutes at approximately 1000 pm (Beckmann GPR centrifuge), the supernatant removed by suction and the cells re-suspended in 2 ml incubation medium and transferred to a medium sized cell culture bottle to which 10 ml incubation medium were added.

On the fourteenth day in culture the cells were passaged again but this time with 6 ml PBS, 3 ml trypsin/EDTA and 6 ml incubation medium. The cell suspension was centrifuged 5 minutes at 1000 rpm, the supernatant removed by suction and the cells were re-suspended in 6 ml incubation medium, transferred to 3 medium cell culture bottles and 10 ml incubation medium added to each one.

On day 17 a third passage took place to a total of 6 medium cell culture bottles and on day 24 a fourth passage to a total of 12 medium cell culture bottles. Now at the latest all primary cells except for the stem cells had been removed from the cell culture.

The stem cells can be cultivated further and passaged and seeded as often as desired. The seeding preferably takes place at a density of 2-4×10⁵ cells/cm² in the incubation medium. The introduction and further cultivation in the host egg takes place in accordance with the invention in this state again.

### EXAMPLE 3

The isolation and cultivation from exocrine tissue of the parotid gland took place analogously to the pancreas protocol with the following deviations:

1. The exocrine tissue of the parotid gland was a mixture of acinar tissue and tubular tissue.

2. Since salivary glands contain less proteases and amylases than the pancreas, it is possible to store the salivary gland tissue for a while in a refrigerator at approximately 4° C. prior to the workup without the tissue being damaged too much. In the concrete exemplary case the storage time was 15 h and entailed no disadvantageous consequences for the isolation of the desired stem cells.

### EXAMPLE 4

### Preparation of Organoid Bodies

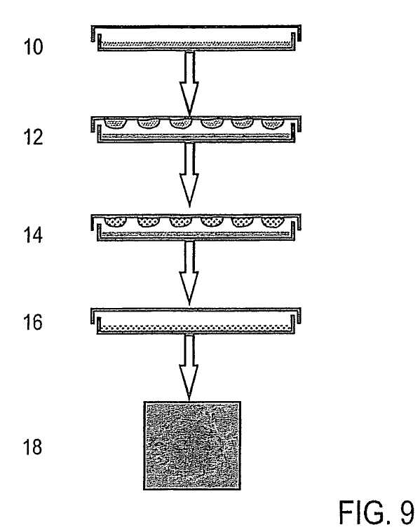

According to the scheme shown in FIG. 9 acinar tissue, preferably of the parotid gland or pancreas, is taken in culture mechanically and enzymatically comminuted for obtaining the stem cells 2 (step 10 in FIG. 9). Contrary to the indications of Bachem et al., Gastroenterol. 115:421-432 (1998), and Grosfils et al., Res. Comm. Chem. Pathol. Pharmacol. 79:99-115 (1993) no tissue blocks are cultivated from which cells are to grow out but rather the tissue was comminuted more strongly under the condition that the cell aggregates of the acini remain very largely intact.

These cells and cell aggregates are cultivated for several weeks in culture vessels. Every 2-3 days the medium is changed, during which all differentiated cells are removed. The cells persisting in culture are undifferentiated cells with an unlimited capacity for division.

In a second step 12 approximately 400 to 800 cells are cultivated in 20 μl medium in hanging drops. To this end, the drops are placed on covers of bacteriological Petri dishes, inverted and placed over the Petri dish filled with medium so that the drops hang downward.

As a result of this type of cultivation the cell aggregates 14 referred to as organoid bodies form within 48 h and are transferred for approximately 6 days into a suspension culture 16. The partial view 18 in FIG. 9 shows a micrograph of such an organoid body.

The organoid bodies growing in suspension culture form new organoid bodies that induce the formation of new organoid bodies even in individual cells. The cells can be frozen as organoid bodies as well as individual cells and retain their vitality and their differentiation capacity.

The following examples 5 and 6 describe in detail two working protocols for producing organoid bodies and differentiated cells.

### EXAMPLE 5

The undifferentiated cells are trypsinated off with a solution of 10 ml PBS, 4 ml trypsin, 8 ml differentiation medium and centrifuged off for 5 minutes. The resulting pellet is re-suspended in differentiation medium in such a manner that a dilution of 3000 cells per 100 μl medium is established. The cells are subsequently well suspended again with a 3 ml pipette.

The cover is removed from bacteriological Petri dishes, which had been previously coated with 15 ml PBS (37° C.) per plate, and inverted. Approximately fifty 20 μl drops are added on a cover with the aid of an automatic pipette. The cover is then rapidly inverted and placed on the Petri dish filled with differentiation medium so that the drops hang downward. The Petri dishes are subsequently carefully placed in an incubator and incubated for 48 h.

Then, the cells that are aggregated in the hanging drops (organoid bodies) are transferred from four covers at a time into one bacteriological Petri dish each with 5 ml incubation medium with 20% FCS and cultivated for another 96 h.

The organoid bodies are now carefully collected with a pipette and transferred into cell culture vessels coated with 0.1% gelatin and containing differentiation medium. In an especially preferred embodiment of the method 6 cm Petri dishes coated with 0.1% gelatin into which 4 ml differentiation medium had been placed and that were subsequently each loaded with 6 organoid bodies are used as culture vessel. Another preferred culture vessel are chamber slides coated with 0.1% gelatin into which 3 ml differentiation medium had been placed and that were subsequently each loaded with 3-8 organoid bodies. In addition, 24-well microtiter plates can also be used that are coated with 0.1% gelatin and into which 1.5 ml per well differentiation medium had been placed and that are subsequently loaded with 4 organoid bodies each.

Cultivated in this manner, the ability of the cells in the organoid bodies to differentiate is activated and the cells differentiate into cells of the three germ layers mesoderm, entoderm and ectoderm. The cells can be stored and cultivated as organoid bodies as well as individual cells and retain their pluripotency.

The differentiation may take place as a pre-differentiation outside of the host egg or be activated by the addition of the cited differentiation factors during the cultivation in the host egg. So far, i.a. smooth muscle cells, neurons, glia cells, epithelial cells, fat cells, cardiac cells, kidney cells, fibroblasts (e.g., skin- and tendon fibroblasts), chondrocytes, endocrine and exocrine glandular cells and thus cell types of all three germ layers have been demonstrated.

### EXAMPLE 6

Stem cells were preferably used after the 42nd day of cultivation for the cultivation in the host egg. The use of stem cells after the 3rd or 4th passage or of cells that had been stored 12-18 months at the temperature of liquid nitrogen was also possible without problems.

At first, the cells were transferred into differentiation medium having the above-indicated composition and adjusted to a density of approximately 3×10⁴ cells/ml, e.g., by trypsin treatment of a stem cell culture in nutrient medium, 5-minute centrifugation at 1000 rpm and re-suspension of the pellets in differentiation medium and dilution to the extent required.

Subsequently, approximately 50 20 μl drops (600 cells/20 μl) were placed with a 20 μ1 pipette on the inside of the cover of a bacteriological Petri dish (plugged tips) and the cover carefully turned over onto the Petri dishes filled with PBS so that the drops hang downward. A new tip was used for each cover. The Petri dishes were subsequently carefully placed in an incubator and incubated 48 h at 37° C.

Then the cells aggregated in the hanging drops, the organoid bodies, were transferred from four covers at a time into one bacteriological Petri dish each with 5 ml incubation medium with 20% FCS (hold cover obliquely and wash the OBs off with approximately 2.5 ml nutrient medium) and cultivated for another 5-9 days, preferably 96 h.

The organoid bodies were now carefully collected with a pipette and transferred into cell culture vessels coated with 0.1% gelatin and containing differentiation medium. The OBs now proliferated and grew into partly individual cell colonies that were able to be proliferated, individualized and proliferated again. In an especially preferred embodiment of the method 6 cm Petri dishes coated with 0.1% gelatin into which 4 ml differentiation medium had been placed and that were each loaded with 6 organoid bodies were used as culture vessel. Another preferred culture vessel were chamber slides coated with 0.1% gelatin into which 3 ml differentiation medium had been placed and that were subsequently each loaded with 3-8 organoid bodies, and Thermanox plates (Nalge Nonc International, USA) for electron microscopic studies. Another alternative were 24-well microtiter plates that had been coated with 0.1% gelatin and into which 1.5 ml differentiation medium had been placed per well and that were subsequently loaded with 4 organoid bodies each.

The organoid bodies or tissue bodies were subsequently suspended in the differentiation medium. The transfer into a chicken egg took place in the suspended state. A drawn-out glass capillary that had been rendered hydrophobic was used for this purpose. A suspension volume was adjusted that was approximately 2 to 20 times greater than the volume of the cell material. The further cultivation in the chicken egg took place under gassing with an air-CO₂ mixture (CO₂ content: 5%).

### EXAMPLE 7

In order to isolate and cultivate human adult stem cells, human tissue was obtained from adult patients immediately after a surgical intervention and immediately worked up. Healthy tissue was separated from the surgically removed tissue, e.g., pancreatic tissue or salivary gland tissue, and taken up in digestion medium. This tissue was then prepared analogously to the protocol described for the rat and the stem cells were isolated and cultivated in an analogous manner.

### EXAMPLE 8

When introducing cell material stemming from fish of a first species into fish eggs of another species, one may start, e.g., from stem cells obtained from the pancreas of eels. These stem cells are implanted according to the above-described techniques into trout or carp eggs and cultivated in them. As a result, eels can be hatched out, which was previously not possible in cultivation methods since eels only spawn in the deep sea.

The features of the invention disclosed in the above description, the claims and the drawings can be significant individually as well as in combination for the realization of the invention in its different embodiments.

1-53. (canceled)

54. A method for cultivating stem cells, comprising:
introducing cell material comprising at least one stem cell of a donor organism into a host egg including a nutrient reservoir; and
cultivating the cell material in the host egg.

55. The method according to claim 54, wherein said host egg does not stem from a mammalian organism.

56. The method according to claim 55, wherein said organism from which said host egg originated is of a class selected from the group consisting of birds, fish, amphibians, reptiles and insects.

57. The method according to claim 54, wherein said host egg is an unfertilized host egg.

58. The method according to claim 54, wherein said host egg is a fertilized host egg.

59. The method according to claim 58, further comprising removing a germ disk from the host egg prior to said step of introducing the cell material into said host egg.

60. The method according to claim 58, further comprising deactivating a germ disk from the host egg prior to said step of introducing the cell material into said host egg.

61. The method according claim 54, wherein the at least one stem cell is an adult stem cell.

62. The method according to claim 61, wherein the adult stem cell stems from an exocrine gland of the donor organism.

63. The method according to claim 54, wherein the at least one stem cell is a non-human embryonic stem cell of the donor organism.

64. The method according to claim 54, wherein the cell material introduced into the host egg comprises at least one aggregate of stem cells that forms a cell body.

65. The method according to claim 54, wherein the cultivation of the cell material in the host egg comprises a differentiated growth of the cell material.

66. The method according to claim 65, wherein the differentiated growth of the cell material is influenced at least one condition selected from the group consisting of material, mechanical or thermal growth conditions.

67. The method according to claim 66, further comprising providing an injection of a cultivation substance with at least one differentiation factor and/or at least one growth factor into the host egg.

68. The method according to claim 54, further comprising measuring during cultivation at least one property of the cell material.

69. The method according to claim 68, wherein the property measured is characteristic for a vitality state of the cell material.

70. The method according to claim 54, further comprising terminating said cultivation of the cell material in the host egg when a predetermined cultivation state is been reached.

71. The method according to claim 70, wherein the cultivation state comprises at least one from the cultivation states consisting of a cultivation duration, a size, and a degree of differentiation of the cultivated cell material.

72. The method according to claim 54, further comprising arranging the cell material in the host egg at a position in the host egg corresponding to the position of the germ disk.

73. The method according to claim 54, further comprising arranging the cell material in the host egg in a cultivation bubble containing a cultivation substance.

74. The method according to claim 72, wherein the cultivation substance in the cultivation bubble is continuously exchanged during the cultivation of the cell material in the host egg.

75. The method according to claim 72, wherein the cultivation substance in the cultivation bubble is repeatedly exchanged in accordance with predetermined time intervals.

76. The method according to claim 54, further comprising exchanging at least a portion of a natural nutrient reservoir of said host egg by a cultivation substance.

77. The method according to claim 76, wherein said exchanging takes place substantially during the cultivation of the cell material in the host egg.

78. The method according to claim 76, wherein said exchanging comprises introducing drops of the cultivation substance into the nutrient reservoir of the host egg.

79. The method according to claim 78, wherein introducing drops of the cultivation substance comprises introducing the drops in a membrane-surrounded state into the host egg.

80. The method according to claim 76, wherein exchanging at least a portion of a natural nutrient reservoir of said host egg by a cultivation substance comprises completely replacing at least one of the group consisting of egg white and egg yolk of the host egg by the cultivation substance.

81. The method according to claim 54, further comprising producing during cultivation of the cell material in the host egg at least one gradient selected from the gradients consisting of a material gradient and a temperature gradient.

82. The method according to claim 54, wherein geometric boundary conditions for the cultivation of the differentiation medium are given with at least one solid.

83. The method according to claim 82, wherein the solid is formed by a carrier particle that forms a substrate for the stem cells during the cultivation.

84. The method according to claim 83, wherein the cell material is introduced in a compound with the carrier particle into the host egg.

85. The method according to claim 82, wherein the solid is formed by a substance hardened in the host egg.

86. A cell culture comprising:
a host egg including a nutrient reservoir; and
cell material comprising at least one stem cell of a donor organism, wherein said cell material is arranged in the host egg.

87. The cell culture according to claim 86, wherein the cell material does not stem from a mammalian organism.

88. The cell culture according to claim 87, wherein the cell material stems from an organism having a class selected from the group consisting of birds, fish, amphibians, reptiles and insects.

89. The cell culture according to claim 86, wherein the host egg is an unfertilized host egg.

90. The cell culture according to claim 86, wherein the host egg contains a deactivated germ disk.

91. The cell culture according to claim 86, wherein the host egg contains no germ disk.

92. The cell culture according to claim 86, wherein the at least one stem cell is an adult stem cell.

93. The cell culture according to claim 86, wherein the at least one stem cell is a non-human embryonic stem cell of the donor organism.

94. The cell culture according to claim 86, wherein the cell material comprises at least one aggregate of stem cells that forms a cell body.

95. The cell culture according to claim 86, wherein a cultivation substance with at least one differentiation factor and/or at least one growth factor is arranged in the host egg.

96. The cell culture according to claim 86, wherein the host egg includes a cultivation bubble containing a cultivation substance in which the cell material is arranged.

97. The cell culture according to claim 86, wherein at least a part of the nutrient reservoir is formed by a cultivation substance introduced from outside said host egg into the host egg.

98. The cell culture according to claim 86, wherein the host egg contains at least one solid for giving geometric boundary conditions for the cultivation of the cell material.

99. The cell culture according to claim 98, wherein the at least one solid comprises at least one selected from the group consisting of at least one carrier particle and at least one substance hardened in the host egg.

100. A cell culture device comprising:
an incubator device with an incubator chamber designed to receive a host egg with a natural nutrient reservoir.

101. The cell culture device according to claim 100, further comprising an injection device for introducing a liquid or gaseous substance into the host egg.

102. The cell culture device according to claim 101, wherein the injection device comprises a suspension injector.

103. The cell culture device according to claim 101, wherein the injection device comprises a liquid injector.

104. The cell culture device according to claim 101, wherein the injection device comprises a gas injector.

105. The cell culture device according to claim 100, further comprising a temperature-adjusting device.

106. The cell culture device according to claim 100, further comprising a humidifier.

107. The cell culture device according to claim 100, further comprising a measuring device.

108. The cell culture device according to claim 107, wherein the measuring device comprises a probe arranged in the incubator device in the host egg.

109. The cell culture device according to claim 100, further comprising a drive device for moving the incubator device.

110. A method comprising use of an egg of a host organism for the cultivation of biological cells that do not stem from the host organism.

111. The method of claim 112, wherein said biological cells are stem cells.

112. A method comprising using an egg as an incubator device for biological cells inserted from outside said egg into the egg.

113. The method of claim 112, wherein said biological cells are stem cells.
